(19) United States
(12) Patent Application Publication
(10) Pub. No.: US 2025/0134402 A1
(43) Pub. Date: May 1, 2025

(54) APPARATUS AND METHOD FOR CONTACTLESSLY SENSING BIOLOGICAL SIGNAL AND RECOGNIZING USER HEALTH INFORMATION USING SAME

(71) Applicant: ELECTRONICS AND TELECOMMUNICATIONS RESEARCH INSTITUTE, Daejeon (KR)

(72) Inventors: Hee Sook SHIN, Daejeon (KR); Yong Ho LEE, Daejeon (KR); Youn Hee GIL, Daejeon (KR); Seong Min BAEK, Daejeon (KR); Cho Rong YU, Daejeon (KR); Sung Jin HONG, Daejeon (KR)

(73) Assignee: ELECTRONICS AND TELECOMMUNICATIONS RESEARCH INSTITUTE, Daejeon (KR)

(21) Appl. No.: 18/646,570

(22) Filed: Apr. 25, 2024

(30) Foreign Application Priority Data
Oct. 25, 2023 (KR) 10-2023-0143824

(51) Int. Cl. A61B 5/024; A61B 5/00; A61B 5/01; G06T 5/70; G06T 7/00

(52) U.S. Cl. CPC A61B 5/02416; A61B 5/01; A61B 5/7203; A61B 5/7485; G06T 5/70; G06T 7/0012; G06T 2207/10016; G06T 2207/10024; G06T 2207/10048; G06T 2207/30201

(57) ABSTRACT

Proposed is an apparatus and method for contactlessly sensing a biological signal and recognizing a user's physiological information using same. The operating method of the sensing apparatus for contactlessly sensing biological signals may include a process of performing an initial setting in a contactless sensing apparatus, a process of obtaining video data from the contactless sensing apparatus, a process of removing noise from the video data, a process of extracting only a region of interest from the video data, and a process of estimating information on biological activity by analyzing the region of interest.

### CROSS REFERENCE TO RELATED APPLICATION

The present application claims priority to Korean Patent Application No. 10-2023-0143824, filed on Oct. 25, 2023, the entire contents of which are incorporated herein for all purposes by this reference.

### BACKGROUND

### Technical Field

The present disclosure generally relates to an electronic apparatus and, more particularly, to an apparatus and method for contactlessly sensing a biological signal and recognizing a user's physiological information using same.

### Description of the Related Art

In augmented reality (AR) and virtual reality (VR) technologies, there have been many attempts to increase a sense of immersion and presence by acquiring various information on a user. As an example, there has been an attempt to improve a sense of immersion by identifying the movements of a user's joints and muscles. Apart from such external information, there have been attempts to obtain user information from internal biological signals. In particular, heart-related information has been widely used in the healthcare field, and has become an important factor in measuring a user's mental health state beyond his or her physical health state.

### SUMMARY

On the basis of the discussion above, the present disclosure provides an apparatus and method for contactlessly sensing biological signals and recognizing a user's physiological information using same.

In addition, the present disclosure provides an apparatus and method for obtaining a user's biological signals.

In addition, the present disclosure provides an apparatus and method for obtaining information about a user's emotional states and physical activities.

In addition, the present disclosure provides an apparatus and method for sensing a user's biological signals while being attached to a user's head-mounted device that displays a virtual space.

According to various exemplary embodiments of the present disclosure, an operating method of a sensing apparatus for contactlessly sensing biological signals may include a process of performing an initial setting in a contactless sensing apparatus, a process of obtaining video data from the contactless sensing apparatus, a process of removing noise from the video data, a process of extracting only a region of interest from the video data, and a process of estimating information about biological activity by analyzing the region of interest.

According to various exemplary embodiments of the present disclosure, a sensing apparatus for contactlessly sensing biological signals may be in an integrated form that is included as part of a display device worn on the user's face and may include a memory, a communication device, and a processor which is operably connected to the memory and the communication device, wherein the processor performs an initial setting in the contactless sensing apparatus, obtains video data from the contactless sensing apparatus, transmits through the communication device the obtained video data to a computing device that performs analysis, removes noise from the video data in the computing device, extracts only a region of interest of the video data at the computing device, and estimates information about biological activity by analyzing the region of interest.

According to various exemplary embodiments of the present disclosure, a sensing apparatus for contactlessly sensing biological signals may be a stand-alone type to independently be operable apart from a display device worn on a user's face and may include a memory, a communication device, and a processor that is operably connected to the memory and the communication device, wherein the processor performs an initial setting in the contactless sensing apparatus, obtains video data from the contactless sensing apparatus, removes noise from the video data, extracts only a region of interest of the video data, and estimates information about biological activity by analyzing the region of interest.

An apparatus and method according to various exemplary embodiments of the present disclosure may improve a user's immersion and presence in a virtual space by sensing biological signals through a contactless sensor.

The effects that are obtained from the present disclosure are not limited to the effects described above, and other effects not mentioned will be clearly understood by those skilled in the art to which the present disclosure belongs from the following description.

### BRIEF DESCRIPTION OF THE DRAWINGS

FIG. 1 shows an example of being disposed and operated in an HMD device according to an exemplary embodiment of the present disclosure.

FIGS. 2A and 2B show an example in which an integrated sensor apparatus and a stand-alone sensor apparatus are interlocked with an HMD according to an exemplary embodiment of the present disclosure.

FIG. 3 shows a function configuration of an apparatus according to an exemplary embodiment of the present disclosure.

FIG. 4 shows an operation configuration diagram of an apparatus in detail according to an exemplary embodiment of the present disclosure.

FIG. 5 shows an operating method of an apparatus according to an exemplary embodiment of the present disclosure.

FIG. 6 shows a structure diagram of an apparatus according to various exemplary embodiments of the present disclosure.

FIGS. 7A and 7B show a comparison between a PPG method and an rPPG method according to various exemplary embodiments of the present disclosure.

FIGS. 8A and 8B show an example of an actual product of a PPG method and an rPPG method according to various exemplary embodiments of the present disclosure.

### DETAILED DESCRIPTION OF THE DISCLOSURE

Terms used in the present disclosure are used only to describe specific exemplary embodiments and may not be intended to limit the scope of other exemplary embodiments. Singular expressions may include plural expressions unless the context clearly indicates otherwise. Terms used herein, including technical or scientific terms, may have the same meaning as that generally understood by those of ordinary skill in the art described herein. Terms used in this disclosure that are defined in common dictionaries are to be construed as having the same or similar meaning as having in the context of the relevant technology, and are not interpreted as ideal or excessively formal unless explicitly defined in the present disclosure. In some cases, even terms defined in the present disclosure cannot be interpreted to exclude exemplary embodiments of the present disclosure.

In various exemplary embodiments of the present disclosure described below, a hardware approach is described as an example. However, various exemplary embodiments of the present disclosure do not exclude a software-based approach since various exemplary embodiments of the present disclosure include techniques that utilize both hardware and software.

In addition, in the detailed description and claims of the present disclosure, “at least one of A, B, and C” may mean “only A”, “only B”, “only C”, or “any combination of A, B, and C.” In addition, “at least one of A, B, or C” or “at least one of A, B, and/or C” may mean “at least one of A, B, and C.”

The present disclosure relates generally to a head-mounted display (HMD) and, more particularly, to an apparatus and method for contactlessly sensing biological signals and recognizing a user's physiological information using the same. Specifically, the present disclosure may explain a technology for recognizing a user's physiological information using a contactless sensor apparatus that is attached to a user's head-mounted device for displaying a virtual space in order to recognize information about a user's emotional states and physical activities in the virtual space. According to an exemplary embodiment, the user's head-mounted device may include a form of head-mounted display, a form of glasses, and a form of a headset.

Augmented reality (AR) and virtual reality (VR) technologies may visualize virtual spaces and objects to users through head-mounted display (HMD) devices and provide interaction therewith through interface devices or the user's body movements, thereby increasing the sense of immersion in the virtual space.

Since the sense of immersion and reality like facts increases the user's sense of existence in virtual spaces, AR and VR technologies may seek to provide effective information that increases a sense of existence on the basis of a user's more physiological information. For this, not only may the position of the user's hands and head be tracked using sensors such as inside-out tracking cameras, accelerometers, gyroscopes, and magnetometers located on the HMD, but facial expressions may be also estimated from the minute movements of the user's facial muscles and eye tracking data while the HMD is worn.

Such information may be mainly obtained by sensing and analyzing externally visible data such as the movements of a user's joints and muscles and most of the data may be collected from HMD devices and controllers which are the basic equipment for actualizing virtual reality technologies.

In recent years, there has been an increase in attempts to obtain information about a user not only from externally revealed information but also internal from biological signals. Information such as heart rate, oxygen saturation level with pulse oximeter, and skin temperature may become data that estimate the user's physical and mental state. In particular, heart-related data has traditionally been widely used in the healthcare field and may be not only basic information for measuring the user's physical health state, but also an important factor in measuring the user's mental health state such as stress and emotional states.

Data related to the heart activity, such as heart rate (HR) and heart rate variability (HRV) may be mainly obtained by PPG, that is, a measuring method called photoplethysmography. This method may estimate the state of the heart activity by measuring variations in intravascular blood volume depending on the relaxation and contraction of the heart by using optical properties such as reflectance, absorption, and transmittance of light with respect to biological tissue. Therefore, the PPG sensor device may be provided in the form of a skin-contact sensor that easily and stably observes blood vessels under the skin. A skin-contact type of wearable device has been developed in a way that the reflective method for measuring the reflected light is shaped to be a band-type form factor worn on the wrist and the transmissive method for measuring the transmitted light is shaped to be a thimble form factor worn on the fingertips.

Recently, a technology called rPPG, or remote photoplethysmography has been utilized to relieve the inconvenience of wearing such a device and to obtain biological information about the heart during a user's more free activities. This is a method of obtaining information about heart activities from videos using computer vision technology based on a general camera or an image sensor. A user's face may be recognized from remotely collected videos and a specific skin area of the face may be extracted in order to estimate blood volume changes from changes in the videos. In general, rPPG may be mainly applied to applications that use cell phone cameras or webcams and it may be known that the skin images of the forehead and cheeks among a user's face provide good data. This may seem to be a result of limiting to a region where it is easy to obtain videos in consideration of the camera's attachment position and the user's movements.

Recently, virtual reality technology may be widely used not only for mental health purposes such as VR therapy and psychotherapy, but also for games and training, and information about heart rate may be obtained for the purpose of identifying a user's physiological information in a virtual environment.

Conventionally, a separate measuring device having a PPG sensor may be attached to the wrist and chest for use to sense a user's heart activity information in related virtual contents or services. This method may be not only different from the current trend of virtual reality technology development that seeks to be controller-free and introduces many bare-hand interaction technologies, but may also have disadvantages in that it is cumbersome to wear and carry with such a separate device and accuracy is reduced because of the noise measured with the user's movements. As an alternative, signals may be measured from outside using the rPPG method described above, but this has a limitation in that sensing is possible only in a limited space, that is, only within a certain area of the space where the camera is installed, and also there is a problem in that it is not possible to estimate the face area covered by the HMD.

In order to solve these problems, the present disclosure proposes an apparatus and method for sensing signals of the heart activity as data and recognizing information about the user's physical and mental state from the sensed data without additional equipment and without any restriction on space and the user's activity in a state where the user wears the HMD. Specifically, it may be possible to estimate a user's various states through fusion with a plurality of existing sensed data (e.g., rotational motions, directions, movements, gazes, facial muscle movements) which commercial HMDs have.

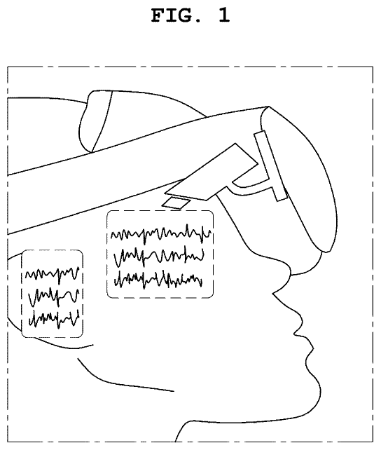

FIG. 1 shows an example of being disposed and operated in an HMD device according to an exemplary embodiment of the present disclosure.

Referring to FIG. 1, the sensor apparatus shown in FIG. 1 may be attached to the side of the HMD and arranged to obtain skin videos, a cheek, or an ear video of the user's side face. As the HMD is fixed and worn on the user's head, it may be possible to obtain close-up skin videos in a non-contact manner regardless of the user's movements. This may provide the convenience of use by allowing only an HMD for displaying a virtual environment to collect biological signals without an additional sensor device being mounted. In addition, it is possible to solve the problem of having to recognize the user's face and continuously track the recognized face as in the existing rPPG method.

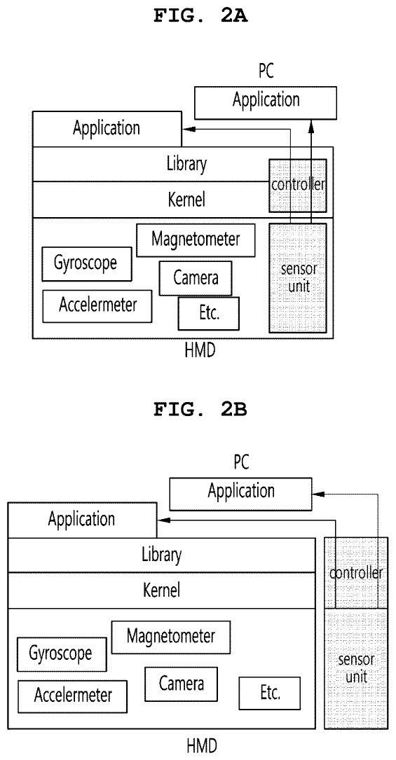

FIGS. 2A and 2B show an example in which an integrated sensor apparatus and a stand-alone sensor apparatus are interlocked with an HMD according to an exemplary embodiment of the present disclosure. FIG. 2A shows an example of an integrated type, and FIG. 2B shows an example of a stand-alone type. Specifically, FIG. 2A shows an integrated type in which a sensor unit and a controller are included in the HMD system, and FIG. 2B shows a stand-alone type in which the sensor unit and the controller are provided separately apart from the HMD system.

Depending on the coupling degree between the sensor apparatus and the HMD, it may be implemented as an integrated type in which the sensor is embedded within the HMD and controlled by the resource management of the HMD's own system, or may be implemented as a stand-alone type that is detachable so that the sensor apparatus can be attached to multiple HMDs for use and can include a sensor controller.

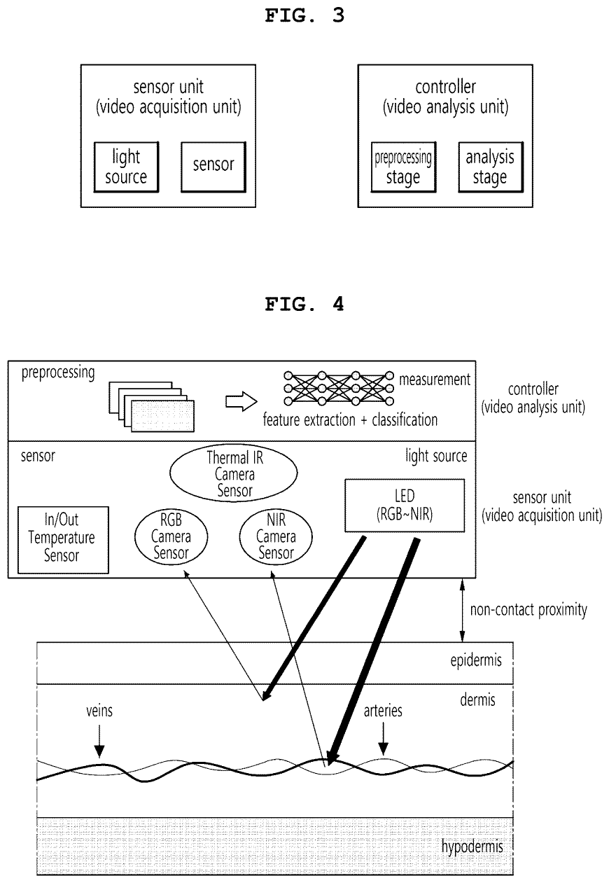

FIG. 3 shows a function configuration of an apparatus according to an exemplary embodiment of the present disclosure.

Referring to FIG. 3, the sensor apparatus may be composed of a sensor unit (video acquisition unit) and a controller (video analysis unit). The video acquisition unit may include a camera sensor that collects skin videos of the side area of the user's face and may additionally include a separate light source depending on a type of camera sensor.

The video acquisition unit of FIG. 3 may correspond to a sensor unit coupled to or separated from the HMD in FIGS. 2A and 2B, and the video analysis unit may operate in the controller coupled to or separated from the HMD of FIGS. 2A and 2B. The controller may be operated to perform a preprocessing stage and an analysis stage.

FIG. 4 shows an operation configuration diagram of an apparatus in detail according to an exemplary embodiment of the present disclosure.

Referring to FIG. 4, a camera sensor may be composed of at least one of a near-infrared (NIR) sensor, a thermal infrared (TIR) sensor, and an RGB sensor. Each sensor may be selectively configured and shaped according to a condition of an application to be applied due to differences in characteristics thereof.

When using an NIR sensor, there may be advantages in that it is easy to analyze in real-time because the data size is relatively reduced as collecting data in the form of a single color video and the analysis results become more reliable because of the high contrast and the low noise. In addition, the NIR sensor may operate robustly against interference from light wavelengths in the visible light range of the HMD's own display. In some cases, a separate IR LED light source may be used together to obtain more accurate data and it may be possible to implement a stable, low-power device because the region and time of the video recordings can be controlled by synchronizing with the NIR sensor. In addition, as the skin transmittance is deep, the NIR sensor may be utilized for the purpose of assisting other sensor types as reference data for the user's facial movements.

When using a thermal IR sensor, it may be possible to measure vascular changes in the face without a separate light source. Vascular changes may be measured from thermal videos by sensing the temperature difference between the vascular part and the non-vascular part due to the contraction and expansion of blood vessels according to the heartbeat.

When using an RGB sensor, changes in RGB values of the skin videos may be measured by measuring the reflected light in the visible light region. An LED light source may be used for more accurate measurement and the sensing time and extraction method may be adjusted in consideration of the visible light output from the HMD when a content display itself has much leakage of the visible light depending on the structural shape of the HMD being worn. For example, data may be obtained at the time when the HMD's output is reduced, or sensor data may be obtained when a certain range of values in a region of RGB values is observed to be above a threshold.

In the video analysis unit of FIG. 4, a preprocessing stage of refining videos to be used for analysis through the necessary pre-processing and a measurement stage of analyzing the waveform changes of the heart activity on the basis of the refined videos may be performed according to the sensor type and analysis method of the obtained videos.

The data transmitted to the video analysis unit may be time-series image data collected by a camera sensor. That is, the difference may be that two-dimensional video data are used rather than linear signal values measured by a single or multiple photodiodes when using a conventional PPG sensor. Therefore, although a stage for pre-processing image data is required, there may be an advantage in that the function of the pre-processing unit is simplified because the sensor is attached in a state where the ROI (region of interest) is already focused, unlike a conventional rPPG method. That is, it is possible to collect and deal with data by simplifying existing stages such as recognizing and continuously tracking a face or extracting a skin area from the face since the location of the sensor is close to the skin area to be extracted in order to collect data.

In the analysis stage, only single data from a camera sensor may be used, or more than one sensor data may be used simultaneously. In particular, NIR sensor data may be used as data for variant reference with respect to facial movements and RGB sensor data may simultaneously be analyzed as major factors. In addition, skin temperature and external temperature may be simultaneously measured with skin and outdoor temperature sensors (in/out temperature sensor in FIG. 4) and be utilized as reference data for data analysis.

In general, noise caused by motion may be in the range of 0 to 10 Hz, so it may be absolutely necessary to remove the corresponding noise in a sensing method that uses frequencies from the visible light to the infrared range.

Therefore, as an example, the proposed may be an arrangement that can analyze RGB sensor data as the main element as utilizing data from NIR sensors, thermal IR sensors, and skin and outdoor temperature sensors (in/out temperature sensor in FIG. 4) as reference signals in the proposed sensor apparatus and the arrangement may enable stable and reliable information extraction even in HMD stand-alone sensor types.

In the video analysis, the waveform may be estimated by calculating the number on the basis of pixels, or output information may be obtained according to the input of video data using an AI model based on deep learning. Other video analysis technologies which are commonly used may be variously applied in the pre-processing and analysis stages.

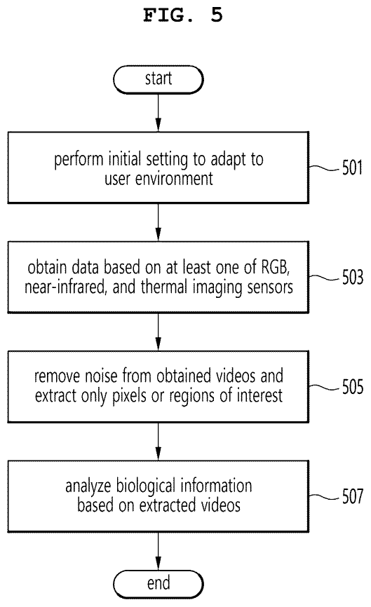

FIG. 5 shows an operating method of an apparatus according to an exemplary embodiment of the present disclosure.

In the operation 501, the contactless sensing apparatus may perform an initial setting to adapt to the user environment. In the operation 501, the contactless sensing apparatus may adjust the focus of the contactless sensing apparatus or operate a light source.

After performing the operation 501, the contactless sensing apparatus may obtain data based on at least one of an RGB sensor, NIR sensor, or Thermal IR sensor (503). In the operation 503 according to an exemplary embodiment, temperature data may be obtained by a temperature sensor, and video data may be obtained through more than one camera sensor.

In the operation 503 according to an exemplary embodiment, the contactless sensing apparatus may include an operation of contactlessly obtaining video data based on at least one region of the cheeks, ears, and forehead regions of the user's face.

In the operation 503 according to an exemplary embodiment, an RGB sensor, NIR sensor, and a thermal IR sensor may be used, and data may be obtained on the basis of at least one of color filter and a wide-angle meter, increasing the accuracy of the sensing data.

The sensor apparatus may remove noise from the obtained video and extract only the pixel or region of interest (505).

The sensor apparatus may analyze biological information based on the extracted video (507). That is, only data necessary for analysis may be extracted from the obtained video through a preprocessing process and then using the extracted data, the heart activity information may be estimated using an AI model or a pixel-based analysis algorithm.

In the operation 507 according to an exemplary embodiment, while being attached to a device mounted on a user's head in the form of an HMD, glasses, or a headset, the sensor apparatus may estimate biological activity information from videos of the ear, cheek, nose, forehead and eye regions from the user's head.

According to an exemplary embodiment, the heart activity information may include biological information such as heart rate, heart rate variability, and the like.

According to the present disclosure, the position of the sensor may be fixed at the position where video acquisition is required, so that the pre-processing stage in the video analysis unit is simplified to perform and the size of the initially obtained data is reduced, thereby being more advantageous for real-time measurement.

In particular, it is easy to synchronize with a large number of existing sensor data (rotational motions, directions, movements, gazes, facial muscle movements) that commercial HMDs have, so that it is possible to estimate the state of a user who is fused with multiple sensors. For example, in estimating a user's emotional state, heart rate and heart rate variability data of the proposed sensing apparatus may be synchronized and used together in real-time along with gaze and facial expression data that is sensed from the HMD.

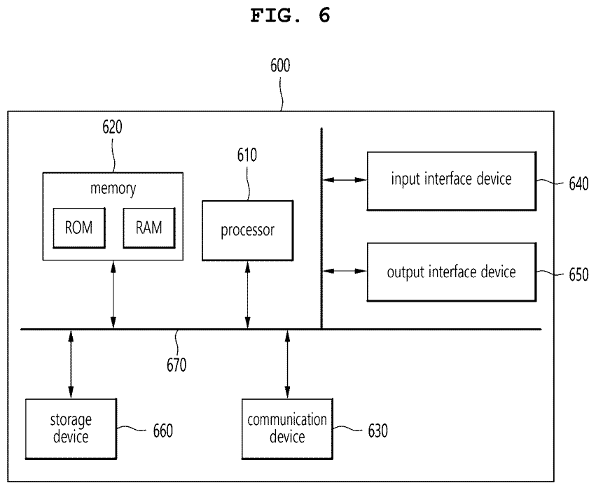

FIG. 6 shows a structure diagram of an apparatus according to various exemplary embodiments of the present disclosure.

Referring to FIG. 6, the sensor apparatus 600 may include at least one processor 610, a memory 620, and a communication device 630 that is connected to a network to perform communications. In addition, the sensor apparatus 600 may further include an input interface device 640, an output interface device 650, a storage device 660, and the like. Each component included in the sensor apparatus 600 may be connected to each other by a bus 670 to communicate with each other.

Simply, each component included in the sensor apparatus 600 may be connected through an individual interface or individual bus centered on the processor 610, rather than through a common bus 670. For example, the processor 610 may be connected through a dedicated interface to at least one of the memory 620, the communication device 630, the input interface device 640, the output interface device 650, and the storage device 660.

The processor 610 may execute a program command stored in at least one of the memory 620 and the storage device 660. The processor 610 may refer to a central processing unit (CPU), a graphics processing unit (GPU), or a dedicated processor on which methods according to exemplary embodiments of the present disclosure are performed. Each of the memory 620 and the storage device 660 may be composed of at least one of a volatile storage medium and a non-volatile storage medium. For example, the memory 620 may be composed of at least one of read-only memory (ROM) and a random access memory (RAM).

Comparable conventional technologies may include technologies that measure the state of heart activity using a PPG sensor, multi-wavelength PPG sensor, and rPPG sensor in a virtual environment.

In general, a PPG attached to the HMD or a multi-wave length PPG sensor type may require minimum contact pressure, so the sensor should be placed on the surface where the HMD comes into contact with facial skin. Therefore, the sensor may be used in a way that is placed on the forehead, or in a way that a separate sensor device is worn on the hands, arms, chest, or fingers. In addition, PPG sensors may provide limited information on the range of signal extraction and the type of signal because the PPG sensor extracts analog signals according to the changes in light intensity through a single or multiple photodiodes. To overcome this, PPG sensors may be sometimes arranged and used in the form of an array, but there remain problems such as the form factor issues due to the increased size, and a limitation of the raw data that a photodiode senses.

Therefore, a type of skin-contact device using such a PPG sensor may be analyzed to have the problem such as the discomfort in wearing and the increased variation depending on the wearing position.

The proposed rPPG, remote photoplethysmographic sensing method, to overcome problems of skin-contact type may be different from the PPG sensor in that the proposed rPPG extracts information on heart activity based on video data. That is, the rPPG may be a method that estimates blood volume changes from the skin images by remotely taking videos using an RGB or IR camera sensor instead of a skin-contact type of PPG sensors and by recognizing the face in the video to extract a specific skin area of the face. This has the advantage of being able to measure remotely and being contactless, but it may have a problem in that it is not easy to find a skin area on the face of a user wearing an HMD.

The contactless sensing apparatus proposed in the present disclosure may seek to solve the problem of conventional technologies by attaching a sensor for wavelengths of RGB, NIR, and thermal IR to the side of the HMD and by immediately obtaining videos of a fixed region of the user's skin. In particular, NIR and thermal IR image data may be utilized as reference data, and AI models that use a combination of these image sensors as a dataset may be effective in estimating information on a user's heart activity.

In addition, it may be possible to collect a user's many biological signals using only an HMD (including a glasses-type display) for displaying a virtual environment without requiring the user to wear additional equipment and, as compared to the existing image-based sensing methods, it may be possible to simplify the pre-processing stage and to reduce the size of the obtained data in the video analysis process, thereby being advantageous for sensing and analyzing data in real-time.

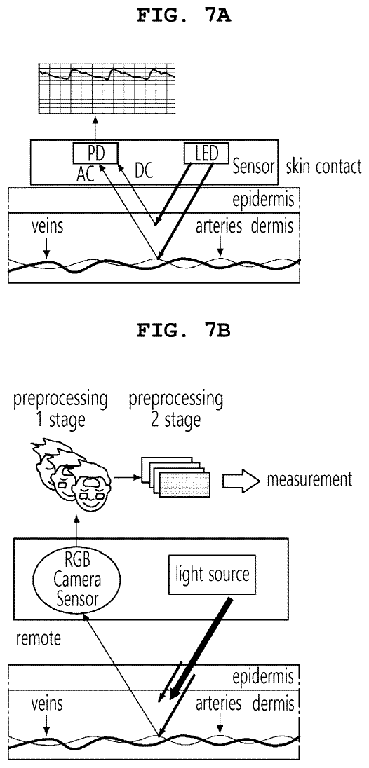

A simple comparison between the PPG method and the rPPG method, which are typically used, is shown in FIGS. 7A and 7B.

FIGS. 7A and 7B show a comparison between the PPG method and the rPPG method according to various exemplary embodiments of the present disclosure. FIG. 7A shows the PPG method, and FIG. 7B shows the rPPG method.

The PPG method in FIG. 7A may sense data through direct contact with the skin.

Unlike the PPG method, the rPPG method of FIG. 7B may not directly contact the skin. For a non-contact method, a light source may be utilized to sense and process data.

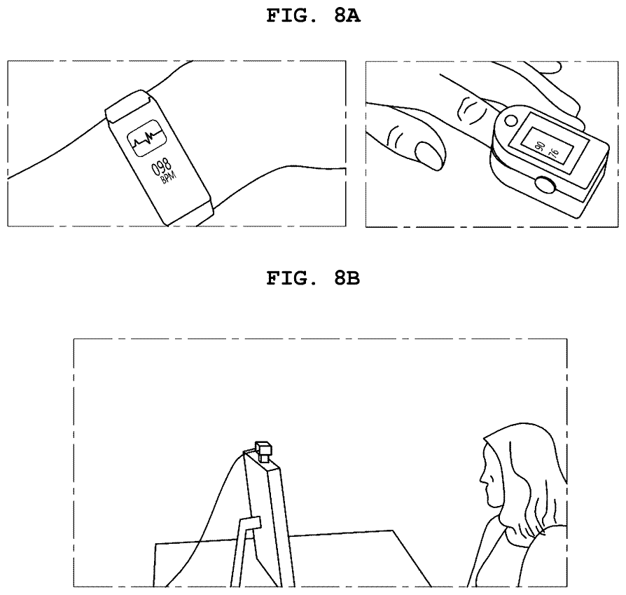

FIGS. 8A and 8B show an example of an actual product of the PPG method and the rPPG method according to various exemplary embodiments of the present disclosure. FIG. 8A shows an actual product of a PPG method, and FIG. 8B shows an example of an actual product of an rPPG method.

FIG. 8A is a PPG method, and it may be seen that there is direct contact with the body.

FIG. 8B is an rPPG method, and it may be seen that a light source is utilized to sense and process data without direct contact with the body.

Methods according to exemplary embodiments described in the claims or specification of the present disclosure may be implemented in the form of hardware, software, or a combination of hardware and software.

When implemented in software, a computer-readable storage medium that stores one or more programs (software modules) may be provided. One or more programs stored in a computer-readable storage medium are configured to be executable by one or more processors in an electronic apparatus. One or more programs may include instructions that enable the electronic apparatus to execute the methods according to the exemplary embodiments described in the claim or specification of the present disclosure. Such programs (software modules, software) may be stored in a random access memory, a non-volatile memory including a flash memory, a read-only memory (ROM), an electrically erasable programmable read-only memory (EEPROM), a magnetic disc storage device, a compact disc-ROM (CD-ROM), a digital versatile disc (DVDs), another type of optical storage device, or a magnetic cassette. Alternatively, programs may be stored in a memory composed of some or all thereof. In addition, there may be multiple of each of these configuration memories.

In addition, programs may be stored in an attachable storage device that is accessible via a communication network such as the Internet, an intranet, a local area network (LAN), a wide area network (WAN) or a storage area network (SAN), or another communication network composed of a combination thereof. Such a storage device may be connected to the device performing an exemplary embodiment of the present disclosure through an external port. In addition, a separate storage device on a communications network may be connected to the device performing the exemplary embodiments of the present disclosure.

In the specific exemplary embodiments of the present disclosure described above, components included in the disclosure are expressed in the singular or plural according to the specific exemplary embodiments presented. However, the singular or plural expressions are selected appropriately for the situation presented for the convenience of explanation, and the present disclosure is not limited to the singular or plural components, and even components expressed in plural may be composed of singular or even components expressed in singular may be composed of plural elements.

Meanwhile, in the detailed description of the present disclosure, a specific exemplary embodiment has been described, but various modifications are possible without departing from the scope of the present disclosure. Therefore, the scope of the present disclosure should not be limited to the described exemplary embodiments and should be determined not only by the scope of the patent claims described later but also by those equal to the scope of the patent claims.

What is claimed is:

1. An operating method of a sensing apparatus for contactlessly sensing a biological signal, the method comprising:
a process of performing an initial setting in a contactless sensing apparatus,
a process of obtaining a video data from the contactless sensing apparatus;
a process of removing noise from the video data;
a process of extracting only a region of interest from the video data; and
a process of estimating a biological activity information by analyzing the region of interest.

2. The method of claim 1, wherein the process of performing the initial setting in the contactless sensing apparatus comprises a process of adjusting a focus of the contactless sensing apparatus or operating a light source.

3. The method of claim 1, wherein the process of obtaining the video data from the contactless sensing apparatus comprises a process of obtaining data based on at least one of an RGB sensor, a near-infrared sensor, or a thermal imaging sensor.

4. The method of claim 1, wherein the process of obtaining the video data from the contactless sensing apparatus comprises a process of contactlessly obtaining the video data based on at least one of a cheek, an ear, and a forehead region of a user's face.

5. The method of claim 1, wherein the process of estimating the biological activity information by analyzing the region of interest comprises a process of estimating the biological activity information based on an AI model or a pixel-based analysis algorithm.

6. The method of claim 1, wherein the biological activity information comprises a heart activity information related to a heart rate or a heart rate variability.

7. The method of claim 1, wherein the sensing apparatus is attached to a display device worn on the user's face and comprises the heart activity information related to the heart rate or the heart rate variability.

8. The method of claim 1, wherein a type of the sensing apparatus is determined on the basis of whether a display device worn on a user's face comprises a controller.

9. A sensing apparatus for contactlessly sensing a biological signal, the apparatus comprising:
a memory;
a communication device; and
a processor operably connected to the memory and the communication device;
wherein the processor
performs an initial setting in the contactless sensing apparatus,
obtains a video data from the contactless sensing apparatus, removes noise from the video data,
extracts only a region of interest from the video data, and
estimates a biological activity information by analyzing the region of interest.

10. The apparatus of claim 9, wherein the processor adjusts a focus of the contactless sensing apparatus or operates a light source in order to perform an initial setting in the contactless sensing apparatus.

11. The apparatus of claim 9, wherein the processor obtains the video data based on at least one of an RGB sensor, a near-infrared sensor, or a thermal image sensor in order to obtain the video data from the contactless sensing apparatus.

12. The apparatus of claim 10, wherein the processor estimates the biological activity information based on an AI model or a pixel-based analysis algorithm in order to estimate the biological activity information by analyzing the region of interest.

13. The apparatus of claim 9, wherein the biological information comprises a heart activity information related to a heart rate or a heart rate variability.

14. A sensing apparatus for contactlessly sensinga biological signal in an integrated form that is included as part of a display device worn on a user's face, the apparatus comprising:
a memory;
a communication device; and
a processor operably connected to the memory and the communication device;
wherein the processor
performs an initial setting in the contactless sensing apparatus,
obtains a video data from the contactless sensing apparatus,
transmits through the communication device the obtained video data to a computing device that performs an analysis,
removes noise from the video data in the computing device,
extracts only a region of interest from the video data in the computing device, and
estimates a biological activity information by analyzing the region of interest in the computing device.
